(19) United States
(12) Patent Application Publication
(10) Pub. No.: US 2004/0161860 A1
(43) Pub. Date: Aug. 19, 2004

(54) Assay with co-immobilized ligands

(75) Inventors: Pascale Richalet-Secordel, Weston, VT (US); Stephen E. Mulholland, Wellington (NZ); Karl Andersson, Uppsala (SE)

Correspondence Address: SEED INTELLECTUAL PROPERTY LAW GROUP PLLC, 701 FIFTH AVE, SUITE 6300, SEATTLE, WA, 98104-7092, US

(73) Assignee: Biacore AB, Uppsala (SE)

(21) Appl. No.: 10/692,284

(22) Filed: Oct. 22, 2003

Related U.S. Application Data
(60) Provisional application No. 60/421,312, filed on Oct. 24, 2002.

(30) Foreign Application Priority Data
Nov. 7, 2002 (SE) 0203216-7

(51) Int. Cl. G01N 33/543

(52) U.S. Cl. 436/518

(57) ABSTRACT

Multivariate type assay methods are disclosed which comprise co-immobilizing different groups of ligands on respective discrete areas of a solid support surface, sequentially contacting the different areas with single or multiple analytes, detecting interactions of the analytes with the ligand groups, and determining therefrom the amount of ligand-binding of each analyte.

### CROSS-REFERENCE TO RELATED APPLICATION

This application claims the benefit of U.S. Provisional Application No. 60/421,312 filed Oct. 24, 2002, and Swedish Patent Application No. 0203216-7 filed Nov. 7, 2002, both of which applications are incorporated herein by reference in their entireties.

### BACKGROUND OF THE INVENTION

1. Field of the Invention

The present invention relates to an improvement in assay methods of the type which comprises contacting an analyte-containing sample with a solid phase surface having immobilized ligand, and determining interaction of the analyte with the ligand, and more particularly to methods where multiple ligands are co-immobilized on the solid phase surface.

2. Description of the Related Art

A variety of analytical techniques are used to characterize interactions between molecules, particularly in the context of assays directed to the detection and interaction of biomolecules. For example, antibody-antigen interactions are of fundamental importance in many fields, including biology, immunology and pharmacology. In this context, many analytical techniques involve binding of a “ligand” (such as an antibody) to a solid support, followed by contacting the ligand with an “analyte” (such as an antigen). Following contact of the ligand and analyte, some characteristic is measured which is indicative of the interaction, such as the ability of the ligand to bind the analyte.

Analytical instrumentation for such assays typically includes biosensors. Such a biosensor usually comprises a sensing surface to which a ligand is immobilized, and binding of an analyte to the sensing surface is detected by the consequential change in a physico-chemical property of the sensing surface. Thus, for example, mass changes at a sensing surface may be sensed to detect ligand binding. One type of methods for determining such changes in the mass at a sensing surface utilizes evanescent wave sensing at an optical surface. Evanescent wave sensing technology based upon surface plasmon resonance (SPR) has been developed for inter alia immunoassay methods. The phenomenon of SPR is well known.

SPR occurs when electromagnetic surface plasmon waves are excited by light in the solid film surface of certain metals or semiconductors. In the case of SPR the metal film is placed between two media of different refractive indices, while for long-range SPR the metal film surrounding media are of similar refractive index. Typically, the metal film, usually silver, copper, aluminium, or gold, is coated on an optically transparent material of fixed refractive index, e.g., glass or plastics, and brought in contact with a liquid sample medium. When monochromatic p-polarized light is totally internally reflected at the glass-metal interface, usually coupled thereto via a prism (Kretschmann arrangement) or a diffraction grating, an evanescent field wave is created at the glass/metal interface which penetrates through the metal. At an angle of incidence specific for the wavelength of the light beam at the glass-metal interface, and specific for the sample medium refractive index, this evanescent field wave may couple to (i.e., transfer the photon energy and momentum to electrons) and excite electromagnetic surface plasmon waves propagating within the metal surface to create an enhanced evanescent wave that penetrates about one light wavelength into the liquid medium. Such plasmon excitation, or resonant light absorption, which is called surface plasmon resonance, or SPR, causes a characteristic drop in the reflected light intensity, which may be detected in real time versus angle of incidence. The “resonance angle” is sensitive to the refractive index of the liquid medium close to the metal layer. Therefore, changes of the refractive index within the penetration depth of the evanescent wave through, for example, a change of mass such as caused by a biomolecular binding event taking place at or near the metal surface may be detected as a corresponding shift in the SPR angle. In general, the refractive index change for a given change of mass concentration at the surface layer is practically the same for all proteins and peptides, and is similar for glycoproteins, lipids and nucleic acids.

WO 93/25910 discloses an SPR-based assay that comprises co-immobilizing to the same sensing surface different catching molecules (ligands) each capable of specifically binding to a respective analyte, contacting the sensing surface with sample containing the analytes, and then sequentially contacting specific reagents to the analytes with the sensing surface to thereby determine the binding of analytes to the surface. Due to the co-immobilization of ligands, two or more different analytes may be determined on a single sensing area which will reduce the complexity of the optical and mechanical design (including microfluidics) of the analytical system, and will improve the assay throughput.

The present invention seeks to provide improved assay methods based on the above concept of co-immobilization of catching molecules on solid phase surface areas.

### BRIEF SUMMARY OF THE INVENTION

In brief, the present invention is generally based on using combinations of single or multiple analyte binding to single or multiple ligands immobilized on multiple surface areas in a flexible manner to study binding of molecules in various settings, a salient feature of the invention being a particular way of subgrouping the immobilized ligands and optionally also the analytes. This new approach will increase the throughput of inter alia existing mass sensing instruments, without any modification of the optical sensor or of any mechanical parts thereof. It will also allow the improvement of the quality of binding data through the study of possible ligand-ligand or analyte-analyte interactions simultaneously with the study of the ligand-analyte interactions, which is not possible when using single ligand and single analyte systems.

The present invention therefore, in one aspect thereof, provides a multiple ligand/multiple analyte-based assay method for qualitatively or quantitatively assaying for a plurality of analytes, which method comprises the steps of:

(a) providing a plurality of discrete solid support surface areas,

(b) providing a plurality of different ligands,

(c) defining a first set of different groups of the plurality of ligands, each ligand being present in at least one group,

(d) immobilizing each group of ligands on a different solid support surface area,

(e) providing a plurality of different analytes, each of which is capable of binding to a respective one of the plurality of ligands, at least a major part of the analytes having substantially no cross-reactivity to other ligands,

(f) defining a first set of different groups of the plurality of analytes, each analyte being present in at least one group,

(g) sequentially contacting each group of analytes with the surface areas to bind the analytes in each group to immobilized ligands, and

(h) detecting the interaction of each group of analytes with each group of ligands to determine therefrom the amount of ligand-binding of each analyte.

This aspect of the invention also includes a sandwich assay variant of the above procedure, using defined groups of specific reagents to the different analytes to detect analyte binding to ligands.

Another aspect of the invention relates to a combinatorial multiple ligand/single or multiple analyte assay for studying ligand/ligand interactions and/or analyte/analyte interactions.

Still another aspect of the invention relates to a combinatorial multiple ligand/single analyte assay designed to gain additional binding information and internal controls.

Another aspect of the invention relates to the use of co-immobilization of ligands for screening of conditions for regenerating ligand-supporting surfaces, i.e., conditions at which the binding analyte may be removed without damaging the underlying ligand.

Yet other aspects of the invention relate to the use of the multiple ligand approach for determining ligand immobilization efficiency, for determining analyte concentration, for determining molecular affinity, and for determining molecular interaction kinetics, respectively.

Other advantages, novel features and objects of the invention will become apparent from the following detailed description of the invention when considered in conjunction with the accompanying drawings.

### Nomenclature

The terms “analyte” and “ligand”, which are used herein for the sake of simplicity, are to be construed broadly and generally mean members of any specific binding pair (sbp), one of which (ligand) is immobilized on the solid phase surface, and the other (analyte) is present in a solution contacted with the surface. Ligand therefore includes any capturing or catching agent immobilized on the surface, and analyte includes any specific binding partner thereto.

The term “analyte analogue” as used herein, means a molecule having a similar binding reactivity to an immobilized ligand or capturing molecule as an analyte. The term includes analyte conjugated with another molecule which does not change the binding characteristics of the analyte.

The terms “multiple” and “plurality” as used herein usually refer to two or more.

The term “antibody” as used herein means an immunoglobulin which may be natural or partly or wholly synthetically produced and also includes active fragments, including Fab antigen-binding fragments, univalent fragments and bivalent fragments. The term also covers any protein having a binding domain which is homologous to an immunoglobulin binding domain. Such proteins can be derived from natural sources, or partly or wholly synthetically produced. Exemplary antibodies are the immunoglobulin isotypes and the Fab, Fab′, F(ab′)₂, scFv, Fv, dAb, and Fd fragments.

The term “binding specifically” as used herein means that cross-reactivity (i.e., the binding to other species than the target species) is less than about 1% (calculated on a molar basis).

The term “subgroup” as used herein means, e.g., for a set of ligands or analytes, a group which contains less than the total number of ligands or analytes, respectively.

When the term “sequentially contacting” is used herein in contexts such as “sequentially contacting each group of analytes with the surface areas”, it means that first one group of analytes is contacted with the surface areas, then the next group of analytes is contacted with the surface areas, etc.

### BRIEF DESCRIPTION OF THE DRAWINGS

FIGS. 1a to 1c show the binding level of antibodies (Ab) to their corresponding peptide ligand co-immobilized on the same surface (FIGS. 1a to 1c relating to different discrete surface areas).

FIG. 2 shows the creation of co-immobilized surfaces in three flow cells (FC2-FC4) with each ligand being included in two different subgroups.

FIGS. 3a to 3c show titration curves of mixed analytes binding to their respective ligand corresponding to the first matrix (M01-059) in FIG. 2.

FIGS. 4a to 4c show K_{D} measurement from a 4 parameter fit obtained from measurement of free antibody versus concentration of free peptide in a competition in solution assay, corresponding to the first antibody and peptide subgroup mixture injected over the 3 flow cells of the first matrix (M01-059) in FIG. 2.

### DETAILED DESCRIPTION OF THE INVENTION

As apparent from the above, the present invention generally relates to the application of a multivariate type format to solid phase assays using different subgroups of ligands co-immobilized on separate solid phase areas, usually discrete areas of one or more sensing surfaces of a sensor, such as a biosensor.

According to the invention, it has thus been found that it is possible to expand the throughput and capacity of current sensor instruments by using mixtures of analytes and ligands in various settings. For example, mixtures of multiple ligands (e.g., from 2 to 10 or more) can be immobilized in at least two discrete areas (such as single spots on a sensor surface that is read by a detector) and mixtures of analytes can be sequentially passed over these multiple ligand spots to gain data on all different interactions. This will effectively reduce sample requirements and increase both speed of analysis and capability of existing sensor instruments, thereby multiplying the capability and throughput of an existing instrument by, say, 3-10 fold. The number of ligands and analytes, respectively, in subgroups thereof will vary depending on the particular assay embodiment, but each subgroup usually contains at least two ligands or analytes, and preferably at least three. In some applications, also a group containing all the ligands or analytes may be included.

Preferably, the analytes used will bind specifically to the respective ligands, ie., there is no or insignificant cross-reactivity. However, it will in practice not be possible to provide in all situations analytes that are all specific binders but a few of the analytes will exhibit a certain detectable degree of cross-reactivity to one or more other ligands. It is within the scope of the present invention, however, to permit some degree of such cross-reactivity. Thus, as long as at least a major part of the analytes are specific binders, the binding data obtained by the inventive procedure will still be sufficient for determining the amount of ligand-binding of each analyte, as will also be demonstrated further below.

When no detecting (or enhancing) reagent is used, such as in a direct binding assay, preferably at least about 75%, more preferably at least about 90%, of the analytes are specific binders, whereas in case a detecting reagent (or enhancer) is used (sandwich assay format), it is preferred that at least about 87.5% (or say at least about 90%) of the total of analytes and detecting reagents are specific binders.

For example, assume that 24 different ligands and 24 different analytes are used. For a direct binding assay with at least 75% specific binders, at least 18 of the analytes used would have to bind specifically to their ligands, while no more than 6 analytes would be permitted to be cross-reactive. For a sandwich type assay with at least 87.5% specific binders, on the other hand, at least 42 of the analytes and detecting reagents would be specific binders, whereas no more than 6 would be cross-reactive analytes and/or detecting reagents.

In the present invention binding events at the solid support surface areas may be detected by numerous techniques, including, e.g., measurement of absorbance, methods relying a label, such as a radiolabel, a chromophore, a fluorophore, marker for scattering light, electrochemically active marker (e.g., field effect transistor based potentiometry), electric field active marker (electro-stimulated emission), magnetically active marker, thermoactive marker, a chemiluminescent moiety or a transition metal, as well as, preferably, so-called label free detection systems.

For many applications, detection is conveniently performed with a chemical sensor or a biosensor, which is broadly defined as a device using a component for molecular recognition (e.g., a layer or pattern with immobilized antibodies) in either direct conjunction with a solid state physicochemical transducer, or with a mobile carrier bead/particle being in conjunction with the transducer. While such sensors are typically based on label-free techniques, detecting, e.g., change in mass, refractive index, or thickness for the immobilized layer, there are also sensors relying on some kind of labelling. Typical sensor detection techniques include, but are not limited to, mass detection methods, such as piezoelectric, optical, thermo-optical and surface acoustic wave (SAW) device methods, and electrochemical methods, such as potentiometric, conductometric, amperometric and capacitance/impedance methods. With regard to optical detection methods, representative methods include those that detect mass surface concentration, such as reflection-optical methods, including both internal and external reflection methods, angle, wavelength, polarization, or phase resolved, for example ellipsometry and evanescent wave spectroscopy (EWS), both may include surface plasmon resonance (SPR) spectroscopy, Brewster angle refractometry, critical angle refractometry, frustrated total reflection (FTR), evanescent wave ellipsometry, scattered total internal reflection (STIR), optical wave guide sensors, evanescent wave-based imaging such as critical angle resolved imaging, Brewster angle resolved imaging, SPR angle resolved imaging, and the like. Further, photometric and imaging/microscopy methods based on for example surface enhanced Raman spectroscopy (SERS), surface enhanced resonance Raman spectroscopy (SERRS), evanescent wave fluorescence (TIRF) and phosphorescence may be mentioned, as well as waveguide interferometers, waveguide leaking mode spectroscopy, reflective interference spectroscopy (RIfS), transmission interferometry, holographic spectroscopy, and atomic force microscopy (AFR).

For the purposes of the present invention, it is preferred to use a mass-sensing method, especially evanescent wave-sensing, such as SPR (which phenomenon has briefly described above).

SPR-based analytical systems to which the present invention may be applied are commercially available. One type of such SPR-based biosensors is sold by Biacore AB (Uppsala, Sweden) under the trade name BIACORE®. These biosensors utilize a SPR based mass-sensing technique to provide a “real-time” binding interaction analysis between a surface bound ligand and an analyte of interest.

As mentioned above, a basic feature of the invention is the use of co-immobilized mixtures of ligands. The immobilized ligands may be low molecular weight (LMW) as well as high molecular weight (HMW) molecules or a mix of LMW and HMW molecules. Ligands may, for example, be co-immobilized simultaneously on the same surface via amino groups, sulfhydroxyl groups, guanido groups, imidazole groups, sugar moities or biotin, just to mention a few, or immobilized sequentially on the same surface via the above-mentioned groups or moities, or any combination of the above immobilization methods. For example, protein or peptide ligands may be immobilized via α-carboxyl and ε-amino groups of lysine residues; α-carboxyl or guanido groups of arginine residues; or imidazole groups of histidine residues.

In one embodiment, the present invention relates to determining binding levels of ligands, referred to herein as “immobilization scouting”. For a successful use of biomolecular arrays, particularly protein arrays, immobilization conditions is a stumbling point, it being necessary that the different proteins (ligands) be present at about the same concentration. To this end, different subgroups of a plurality ligands are mixed together and immobilized onto discrete areas of a sensor surface. Then, by sequentially contacting the ligand subgroups with different subgroups of a plurality of analytes, where the analytes in each subgroup are selected such that (preferably) only one analyte in each subgroup reacts with only one ligand subgroup, the relative immobilization efficiency of each ligand (e.g., high, medium, low) may be determined.

By differentiating ligands based on their rate/efficiency of immobilization (usually after correction for molecular weight and concentration), it is possible to prepare multi-ligand surfaces where each ligand is present at roughly the same concentration. It is also possible to prepare surfaces with different subgroups of ligands immobilized to respective discrete surface areas, where the ligands in each subgroup will compete equally with each other for the binding to the surface.

Such defined subgroups may be used in a combinatorial/matrix-like analysis to gain additional data as well as internal controls. Thus, in conventional type bioarray systems, a high degree of redundency is needed to quality-assure data. By carefully selecting the ligand mixes on the surface areas, it is possible to obtain such redundency without increasing the number of surface areas or “spots”. Assume for example in a BIACORE® type instrument, as mentioned above, with four different flow cells (FC1, FC2, FC3, FC4) an exemplary ligand matrix as follows:

| | | | | |
|---|---|---|---|---|
| FC1 | Blank | | | |
| FC2 | Ligands | A | B | C |
| FC3 | Ligands | B | C | D |
| FC4 | Ligands | C | D | E |

Analytes (a, b, c, d, e) are then sequentially passed through the flow cells. This will provide control data against a blank surface, and data for changes of binding effects based on co-ligands immobilized in the same flow cell. As is readily seen, the replicates are n=1 for A and E, n=2 for B and D, and n=3 for C. By making a second matrix frame-shifted by 1 in the matrix, the additional cross-reactive scenarios can be studied, and the “n” for all ligands can be increased to at least 3. In a system with more avaliable surfaces (or spots), such as, for example, a two-dimensional (2-D) array surface, a larger matrix can, of course, be constructed to study more ligand-analyte pairs.

In another embodiment, being a modified variant of the above described immobilization scouting method, possible ligand-ligand interactions may be explored by varying the subgroup content of ligands. Similarly, analyte-analyte interaction may be studied by varying the subgroup content of analytes.

Still another embodiment is more oriented to identify ligand/ligand and/or analyte/analyte altering the binding of ligand-analyte pairs. Assume, for example, that three different ligand-analyte pairs are studied. If all possible combinations of ligands, from one to two and up to three ligands are immobilized at a time on a surface, it is then possible to introduce single analytes sequentially to the surface and to measure binding to respective surface areas. It is also possible to introduce combinations of analytes, such as two by two or three at a time, and decipher the binding events (enhancement or inhibition).

The multiple-ligand concept of the present invention may also be applied to studies of the kinetics of binding events. For example, by studying interactions at several different analyte concentrations, one may determine the association rate constant, dissociation rate constant, association constant and dissociation constant for the interaction as is per se well known in the art.

Another embodiment of the present invention relates to the screening of regeneration conditions for analyte-immobilized ligand pairs. For, e.g., a protein array surface to be used multiple times, it must be capable of being successfully regenerated. That is, the binding analyte must be quickly and completely removed without causing damage to the immobilised ligand. Unlike immobilized DNA or RNA, proteins may vary widely in the sensitivity and response to various regeneration conditions. Typically, this is done by changing the chemical condition, such as by adding acid, base or salt, as is per se known in the art.

Applying the multiple-ligand concept of the invention to the determination of suitable regeneration conditions will permit a large number of analyte-ligand pairs to be screened quickly and efficiently. One approach is to pool a plurality of ligands in subgroups, similarly to the immobilization scouting setting described above, and co-immobilize the subgroups on different surface areas. Subgroups of analytes to the different ligands are then sequentially contacted with the surface areas, (preferably) only one analyte in each subgroup binding to only one ligand in each ligand subgroup. Regeneration solution is then added, followed by sequential addition of the analyte subgroups again to determine if the regeneration solution has affected the binding levels. A reduction in binding level after regeneration can mean that either the surface was not regenerated or the ligand was damaged. In either case, such a regeneration condition is not a viable regeneration choice.

Regeneration solutions may be grouped by type corresponding to the chemical nature or mode of operation (acids, bases, hydrophobes, salts, etc), and the chemical harshness of the regeneration may be gradually increased.

In a variation of regeneration screening as outlined above, the same ligand subgroup is immobilized to a plurality of surface areas, and the analytes to those ligands are then sequentially contacted with the ligands and the binding of each analyte is determined. The surfaces are then regenerated, each surface area with a different regeneration condition. The success of the regeneration is then determined by again sequentially adding the analytes to the surface areas and determining the binding of the different analytes thereto.

The plurality of analytes and co-immobilized ligands concept of the invention is compatible with several different per se conventional assay formats or principles for concentration determination, such as, e.g., direct binding assay, competition in solution assay, sandwich assay, surface competition assay and inhibition assay.

For instance, a direct assay may be performed by co-immobilizing different capturing molecules (ligands) such as, e.g., peptides, to the surface areas which are capable of reacting with the respective analytes such as antibodies. When subgroups of analytes are created such as only one analyte binds to its respective partner per surface, such defined subgroups of mixed analytes may be contacted sequentially with their capturing molecules. The amount of analyte bound to the surface is then proportional to the concentration of analyte in the sample, provided, of course, that the ligand density on the surface is sufficient.

A competition in solution assay may be performed by adding free soluble ligand or free soluble ligand analogue to the analyte in the direct assay setting described above. When subgroups of free soluble ligands or free soluble ligand analogues are created such as only one ligand or ligand analogue binds to its respective analyte per analyte subgroup, such ligand subgroups may be mixed with their respective analyte subgroups and the resulting mixtures may be contacted sequentially with their capturing molecules. The amount of analyte bound to the surface is then inversely proportional to the concentration of free ligand or ligand analogue in the sample.

A sandwich assay may be performed by co-immobilizing different capturing molecules (ligands), such as antibodies, to the surface areas which are capable of reacting with the respective analytes. Each analyte must in this case be bifunctional, i.e., exhibit a second binding site in addition to the first binding site through which the analyte binds to the capturing molecule, the second binding site being identical to or different than the first binding site. Analytes capable of reacting with the capturing co-immobilized molecules may be mixed altogether and the analyte mixture may then be contacted with the capturing surfaces. Subgroups of reagents which are capable of binding to the second binding site of the analytes may be created such as one reagent, for example an antibody, binds to only one analyte captured by a surface area. Such reagent subgroups may then be sequentially contacted with the captured analyte surface areas, the amount of reagent bound being proportional to the amount of captured analytes.

Another example of sandwich assay is when ligands are immobilized singly on single surface as well as co-immobilized in every possible combination by pairs, by triplets, etc, and altogether on a single surface respectively. A complex mixture of analytes, for example serum or cell lysate, may then be contacted with the surfaces. Single reagent binding specifically to the analytes contained in cell lysate or in serum may then be sequentially contacted with the captured analyte surfaces, the amount of reagent bound being proportional to the amount of captured analytes. Information about the ligand environment may also be gathered, e.g., enhancement or inhibition of analyte binding related to the presence of a particular ligand on the surface different than the analyte specific binding partner, as well as identification of free analyte binding partners present in the complex mixture.

In the case of a surface competition assay, subgroups of target molecules may be co-immobilized on the surface areas. When, e.g., mass-sensing is used for the detection, subgroups of high molecular weight (HMW) compounds selected such as only one HMW molecule binds to one target per surface area may then be contacted with the surface together with a mixture of low molecular weight (LMW) compounds that compete with the HMW compound for the binding to the immobilized targets, the HMW compound being either a natural compound or a HMW compound conjugated with a lead substance. While the concentration of HMW compound is kept constant, that of the LMW compound is varied. As is readily understood, the binding response at the surface decreases as the concentration of the LMW compound increases.

In the case of inhibition assays, ligands are co-immobilized on the surface areas. Specific binding partners to the ligands are then added altogether to the surface areas. These specific binding partners may, for example, be contained in serum or in cell lysates, or may be mixtures of recombinant proteins in buffer, etc. Subgroups of analytes may be selected such as one analyte binds to one ligand per single surface area, and may then be contacted sequentially with the surface-bound ligands. The determined amount of each specific analyte will be inversely proportional to the concentration of the respective specific binding partner.

In all the above described assay formats, the detection response may, if desired, be increased by further sequential additions of additional specific reagents which bind to the specific species bound in the preceding detection step.

The contact between the fluid sample medium and the solid support surface areas, preferably the sensing surface areas of a biosensor, may be static, or preferably dynamic, i.e., the sensing surface areas being provided in some kind of flow cell or cells. Flow cells that may be used in the present invention are well known to the skilled person and need not be detailed herein.

Examples of sensing surfaces that may be used in the present invention are described in inter alia our U.S. Pat. Nos. 5,242,828 and 5,436,161 which disclose sensing surfaces capable of selective biomolecular interactions and designed to be used in biosensor systems, particularly systems based upon SPR. These sensing surfaces comprise a film of a free electron metal, preferably silver or gold, having one of its faces coated with a densely packed monolayer of specific organic molecules. To this monolayer a biocompatible porous matrix, e.g., hydrogel, is bound, which matrix is employed for immobilizing suitable ligands for target biomolecules to be determined by the particular biosensor.

Situations where it is of interest to measure binding of multiple molecular pairs and to which the present invention may be applied are, for example, clinical situations requiring the analysis of more than one analyte to make a correct diagnosis or decision, and food as well as environmental analysis where it is of importance to analyse more than one analyte at a time to get a general picture of the situation. Exemplary of such clinical situations are myocardial infarction, cancer treatment, fertility examination and transplantation surgery. The determination of antibiotics in milk is an example of food analysis, and the determination of pesticides in water may be mentioned as an example of environmental analysis. The present invention may also be applied to situations where it is of interest to study the effect of co-immobilizing ligands to the same surface on the binding of analytes, as well as when higher throughput is necessary with the same surface. Exemplary of studying the effect of co-immobilizing ligands on the binding of analytes is the study of signal transduction pathways where the binding of one particular analyte to its specific ligand may be enhanced by the presence of a second ligand and where the same binding might be inhibited by the presence of a third ligand. Exemplary of improving the throughput is when antibodies directed to different targets are co-immobilized as one ligand subgroup on one surface area and when each surface area presents different clones of the same subgroup, allowing multiple epitope mapping on each surface area.

In the non-limiting Examples following below in order to illustrate the present invention further, the measurements are performed using a commercial SPR-based instrument (BIACORE® 3000), which has four flow cells, and commercial sensing surfaces (Sensor Chip CM5™) (both marketed by Biacore AB, Uppsala, Sweden). As mentioned above, BIACORE® instruments are based on surface plasmon resonance (SPR). The analytical data is provided in the form of a sensorgram which plots the signal in resonance units (RU) as a function of time. A signal of 1,000 RU corresponds to the binding of about 1 ng of analyte per mm². A detailed discussion of the technical aspects of BIACORE® instruments and the phenomenon of SPR may be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE® instruments may be found in the aforementioned U.S. Pat. No. 5,492,840. The full disclosures of the above-mentioned U.S. patents are incorporated by reference herein.

### EXAMPLE 1

Immobilization Scouting

Immobilization scouting allows to select subgroups of ligands to be co-immobilized on the same surface. Thirty-six peptides were studied in two experiments for their capacity to bind to the sensor chip surfaces. Buffers and reagents were from Biacore AB (Uppsala, Sweden) aside from those specified otherwise. Immobilization of peptides was performed in the following manner:

A continuous flow of HBS (10 mM Hepes buffer, 0.15 M NaCl, 3.4 mM EDTA, 0.05% Tween), pH 7.4, over the sensing surfaces of the four flow cells of the BIACORE® 3000 instrument was maintained at 5 μl/min. A fraction of the carboxyl groups on the sensing surfaces was activated to form reactive N-hydroxysuccinimide esters by injecting into the instrument 50 μl of a solution containing 0.2 M 1-ethyl-3-(dimethylaminopropyl)carbodiimide hydrochloride (EDC) and 0.05 M N-hydroxysuccinimide (NHS) in water. 50 μl of a solution containing 18 mg of N-phenyldiethanolamine (PDEA) per ml of 0.1 M borate buffer pH 8.5 was injected over the activated surfaces. 10 μl of ethanolamine were injected to deactivate the remaining reactive N-hydroxysuccinimide ester groups. Peptides (Calbiochem, San Diego, Calif., U.S.A.) were randomly grouped by six, pooled (each peptide at 20 μg/ml) in acetate buffer pH 4.5, and injected over the PDEA derivatized matrices. Each group of six was injected over a single surface. Two sensor chips (4 surfaces each, including control surface) were necessary to perform the experiment. Peptides were immobilized through their terminal cystein residue. Free PDEA was deactivated by injection of 20 μl of L-cystein (Sigma, St. Louis, Mo., U.S.A.).

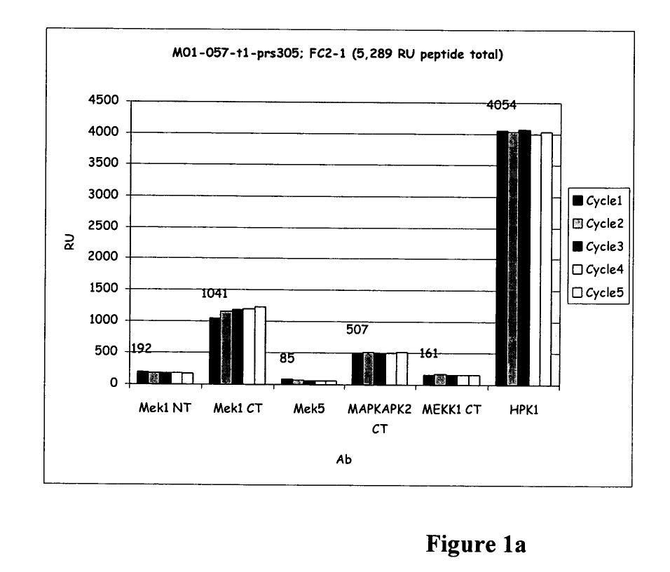

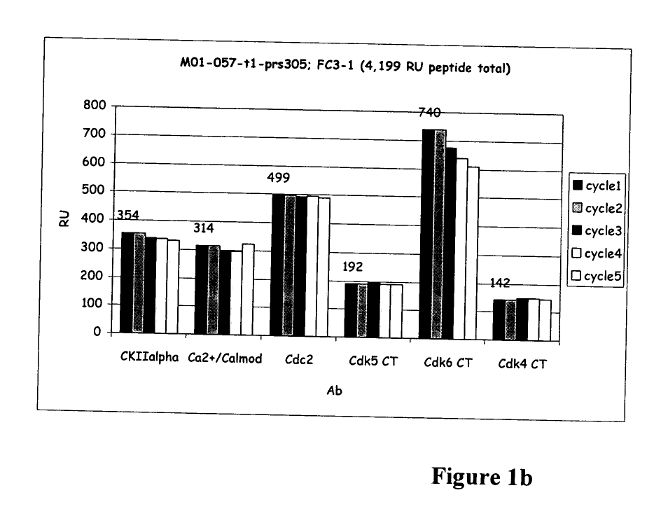

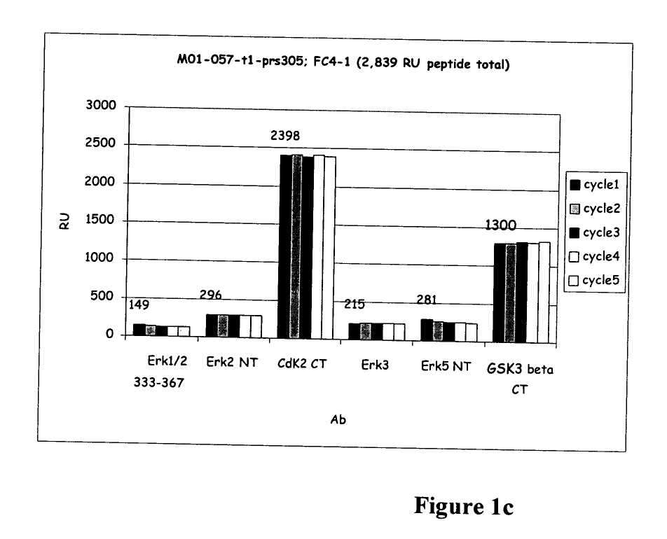

Antibodies (Calbiochem, San Diego, Calif., U.S.A.) (100 nM each) to the respective peptides were pooled by 3 so that only one antibody will bind per surface (flow cell) and were co-injected over the surfaces. Six subgroups were injected over each chip. Each subgroup was injected 5 times. Regeneration was performed with 25 μl of glycine pH 1.5 at a flow rate of 50 μl/min. Binding data from one of the two sensor chips are shown in FIGS. 1a -1c. FIGS. 1a, 1b and 1c correspond to flow cells 2, 3 and 4 of this chip, respectively. Peptides were pooled in four ligand subgroups (1 to 4) based on the binding level of their respective antibody counterparts. Ligand subgroups data are summarized in Table 1 below (the respective subgroup number being indicated within parentheses after each ligand).

TABLE 1

| FC | Peptide 1 | Peptide 2 | Peptide 3 | Peptide 4 | Peptide 5 | Peptide 6 | Total |
|---|---|---|---|---|---|---|---|
| FC2 | Mek1 | Mek1 | Mek5 | MAKKA | MEKK1 | HKP1 | 5,289 |
| | NT (3) | CT (1) | (3) | P2 CT | CT (3) | (1) | |
| | | | | (2) | | | |
| FC3 | CKIIα | Calmodu | Cdc2 (2) | Cdk5 CT | Cdk6 CT | Cdk4 | 4,199 |
| | (2) | line (2) | | (3) | (4) | CT (3) | |
| FC4 | Erk1/2 | Erk2 NT | CdK2 | Erk 3 (2) | Erk 5 NT | GSK3β | 2,839 |
| | 333-67 | (2) | CT (1) | | (2) | CT (1) | |
| | (3) | | | | | | |
| FC2 | Cdk8 NT | DNA PK | Cot NT | SAPKγ | Erk 7-20 | SAPKα | 2,042 |
| | (1) | (3) | (3) | (3) | (3) | (4) | |
| FC3 | SAPKβ | Sek1 (4) | RS6K | P38 | Raf 1 (2) | PSTAI | 5,706 |
| | NT(1) | | CT (4) | HOG1 | | RE(4) | |
| | | | | CT (4) | | | |
| FC4 | Mek6 (2) | c-Mos (3) | Pak3 NT (3) | Weel (2) | Pak1 NT | Tak1 | 7,417 |
| | | | | | (3) | CT (1) | |

Legend:
(1) Group 1 (Ab > 1000 RU)
(2) Group 2 (Ab [500; 1000] RU)
(3) Group 3 (Ab < 500 RU)
(4) Group 4 (Ab < 20 RU or glycine 1.5 not appropriate)

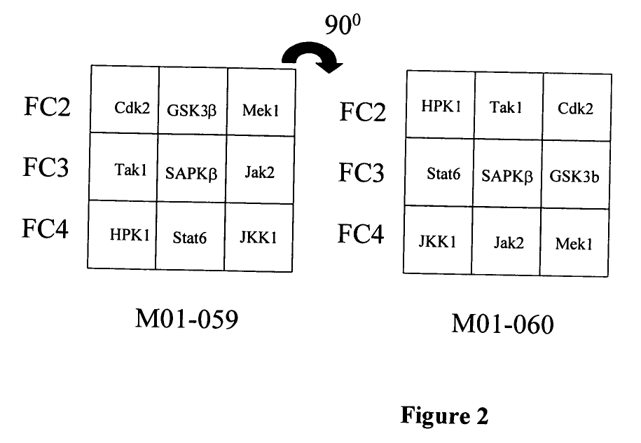

Six peptides from the first group (MEK1-CT, HPK1, CdK2-CT, GSK3β-CT, SAPKβ-NT and Tak1-CT) (see Table 1, group 1) as well as additional Jak2, JKK1 and Stat 6 peptides were pooled by 3 and immobilized on a new CM5 chip, following the same immobilization chemistry as described above. The immobilization experiment was repeated with different subgroups of 3 peptides, as described in FIG. 2. Subgroups of different concentration of antibodies were injected sequentially over the surfaces, subgroups being defined such as only one antibody will bind per surface. Table 2 below summarizes data from the two corresponding sensor chips, i.e., similar concentrations of analyte binding to its particular ligand needed to give a signal comprised in between 100 to 200 RU, and shows that ligands from the same group compete equally for their respective immobilization when pooled and co-immobilized on the same surface. Each ligand from the first group binds the same amount of the same antibody concentration in both settings, which confirms the validity of the approach. In addition, Jak2 and JKK1 that were easily immobilized behave like group 1 ligands, while Stat 6 that was poorly immobilized binds very differently among the two settings (5 fold difference).

TABLE 2

| | |
|---|---|
| M01-059 | M01-060 |

| Peptide | Ab (nM) | RU | Ab (nM) | RU |
|---|---|---|---|---|
| Cdk2 CT | 1.25 | 142 | 2.5 | 97 |
| GSK3β CT | 2.5 | 120 | 2.5 | 135 |
| MEK1 CT | 2.5 | 142 | 2.5 | 150 |
| Tak1 CT | 1.25 | 183 | 2.5 | 106 |
| SAPKβ NT | 0.312 | 174 | 0.312 | 239 |
| Jak2 | 2.5 | 154 | 2.5 | 105 |
| HPK1 | 0.625 | 110 | 0.625 | 101 |
| Stat 6 | 5 | 135 | 1.25 | 179 |
| JKK1 | 1.25 | 125 | 0.625 | 118 |

### EXAMPLE 2

Regeneration Screening

Immobilization scouting was performed as described in Example 1 on antibodies (R&D, Minneapolis, Minn., U.S.A.). They were pooled in subgroups and were co-immobilized, 5 per subgroup, one subgroup per flow cell, on a Sensor Chip CM5, using the following procedure:

A continuous flow of HBS (10 mM Hepes buffer, 0.15 M NaCl, 3.4 mM EDTA, 0.05% Tween), pH 7.4, over the sensing surfaces was maintained at 5 μl/min. A fraction of the carboxyl groups on the sensing surfaces was activated to form reactive N-hydroxysuccinimide esters by injecting into the instrument 50 μl of a solution containing 0.2 M EDC and 0.05 M NHS in water. 50 μl of antibody subgroups (1 μg/ml of each antibody, in acetate buffer pH 4.5) were injected over the surfaces. 10 μl of ethanolamine were injected to deactivate the remaining reactive N-hydroxysuccinimide esters groups.

Regeneration screening: Antibody binding partners (R&D, 50 nM in Hepes buffer) were sequentially injected over the surfaces, followed by one pulse (5 μl at 50 μ/min) of regeneration solution. Table 3 below summarizes regeneration data obtained on flow cell #1, co-immobilized with antibodies to PSA, IFN-γ, TNF-α, IL-12 and IL-1β, and generated by taking a report point after each analyte injection. The numbers correspond to the amount bound to the surface, as expressed in RU, after the regeneration solution (first column) was injected. Shadowed boxes indicate successful regeneration. This experiment does not require a control cell. If, for example, 5 antibodies are immobilized per flow cell (FC), it is then possible to address the regeneration of 20 antibodies simultaneously on the same chip (assuming 4 FC per chip).

TABLE 3

| Regeneration | | | | | |
|---|---|---|---|---|---|
| Buffer | hPSA | hIFN-γ | hTNF-α | hIL-12 | hIL-1β |
| | 219 | 352 | 456 | 169 | 163 |
| 1 mM HCl | 122 | 33 | 270 | 95 | 17 |
| 1 mM HCl | 97 | 26 | 131 | 67 | 14 |
| 1 mM HCl | 78 | 27 | 71 | 51 | 8 |
| 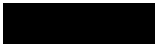 | 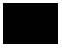 | 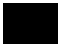 | 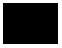 | 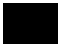 | 12 |
| 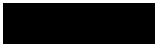 | 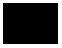 | 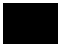 | 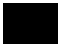 | 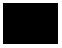 | 14 |
| 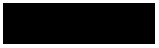 | 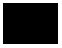 | 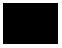 | 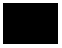 | 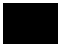 | 14 |
| pH 3 Glycine | 106 | 31 | 226 | 66 | 10 |
| pH 3 Glycine | 86 | 18 | 105 | 49 | 9 |
| pH 3 Glycine | 77 | 16 | 63 | 43 | 7 |
| 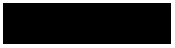 | 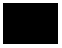 | 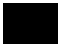 | 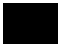 | 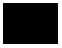 | 18 |
| 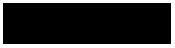 | 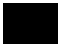 | 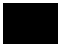 | 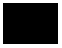 | 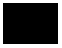 | 16 |
| 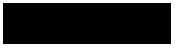 | 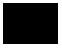 | 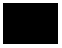 | 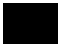 | 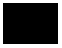 | 16 |
| 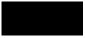 | 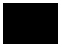 | 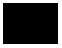 | 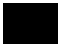 | 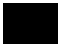 | 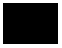 |
| 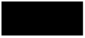 | 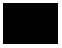 | 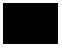 | 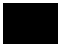 | 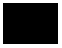 | 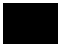 |

Shadowed boxes show successful regeneration

### EXAMPLE 3

A. Affinity and Concentration Measurements of 9 Analyte-Ligand Pairs on a 3×3 Peptide Array, each Ligand being in Two Different Subgroups

Nine peptides (Calbiochem, San Diego, Calif., U.S.A.) were immobilized on two different sensor chips, following the chemistry described in Example 1. Subgroups of 3 peptides are described in FIG. 2.

Subgroups of antibodies (analytes) designed such as only one antibody will bind per surface were sequentially injected (3.1 to 50 nM) over the sensing surfaces for 2 min, using flow rates 10, 25 and 50 μl/min. Samples of affinity and concentration data are shown in Table 4 below. Affinity and concentration were measured using models taking into account bivalent binding and mass transport effects. Upper table shows molecular pairs with similar affinity and concentration values in both settings. The bottom table shows two pairs with different affinity and concentration values from one setting to another, indicating the possible effect of ligand-ligand interactions.

TABLE 4

| | SAPK-β | | HPK1 | | A. Cdk2 | | GSK3-β | | MEK1 | |
|---|---|---|---|---|---|---|---|---|---|---|
| | M01-050- | M01- | M01-050- | M01- | M01-050- | M01- | M01-050- | M01- | M01-050- | M01- |
| | t10 | 061-t4 | t10 | 061-t4 | t10 | 061-t4 | t10 | 061-t4 | t10 | 061-t4 |
| kₐ₁ e5 | 2.2 | 1.1 | 4.2 | 4.2 | 10.7 | 11.2 | 4.9 | 1.6 | 22.9 | 8.1 |
| (M⁻¹s⁻¹) | | | | | | | | | | |
| k_{d1} e-3 (s⁻¹) | 0.4 | 0.4 | 0.3 | 0.3 | 1.2 | 1.7 | 0.4 | 4.0 | 1.2 | 0.6 |
| C (nM) | 44.0 | 44.4 | 20.2 | 23.2 | 10.0 | 8.0 | 4.0 | 11.5 | 2.0 | 8.6 |

B. Solution Affinity Measurements of 9 Analyte-Ligand Pairs on a 3×3 Peptide Array

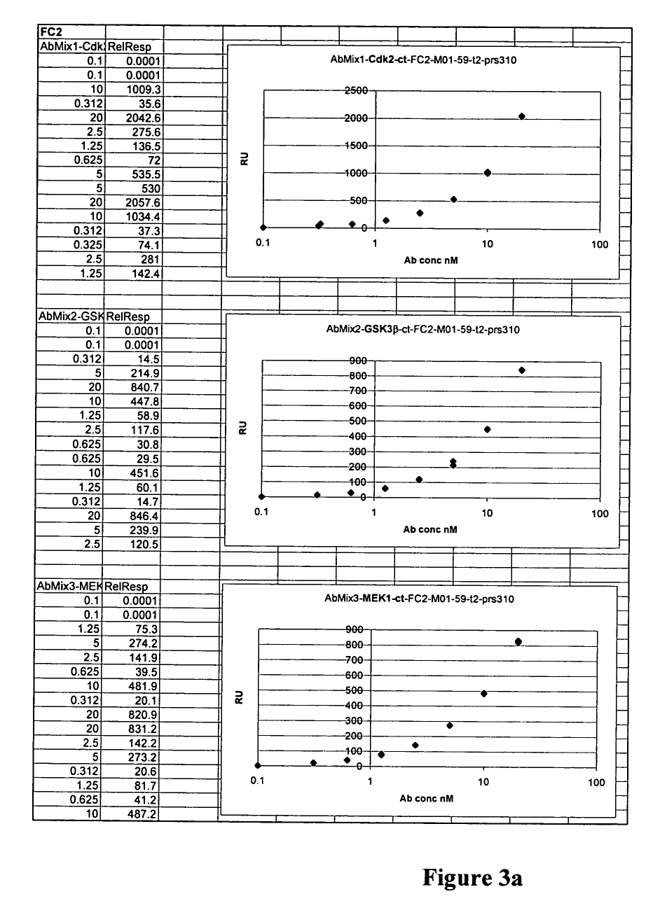

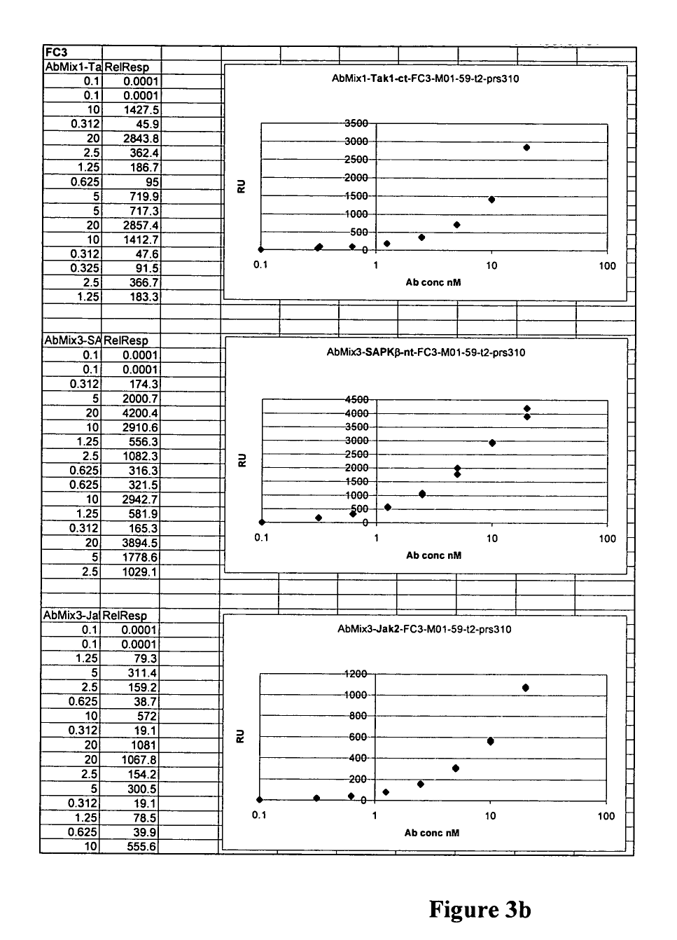

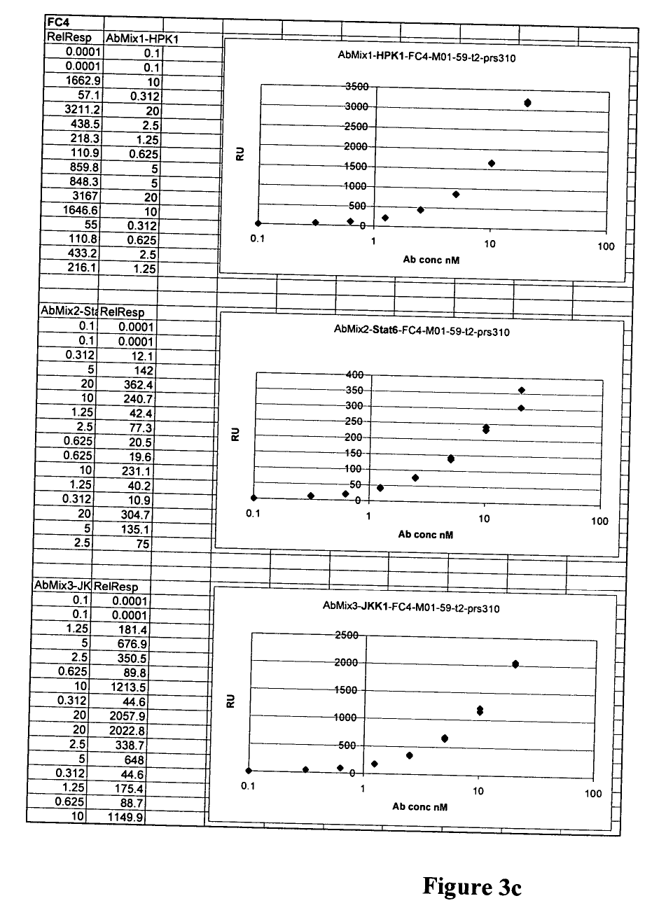

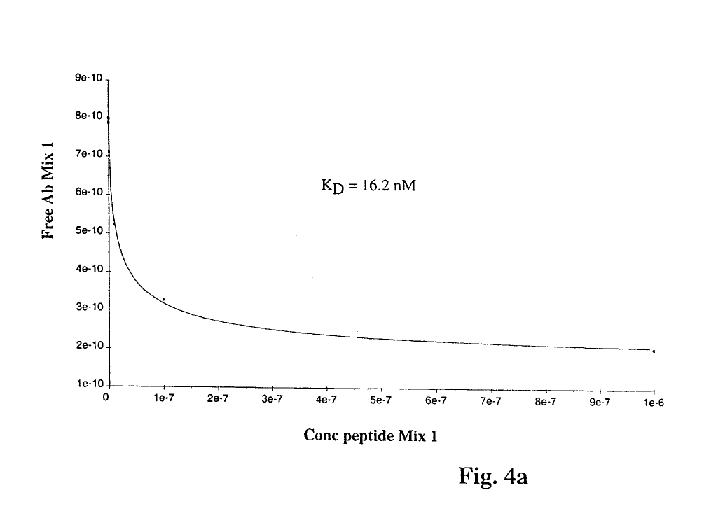

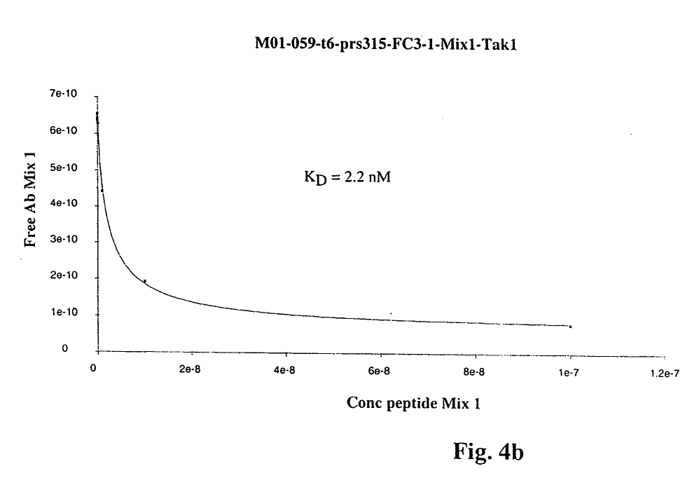

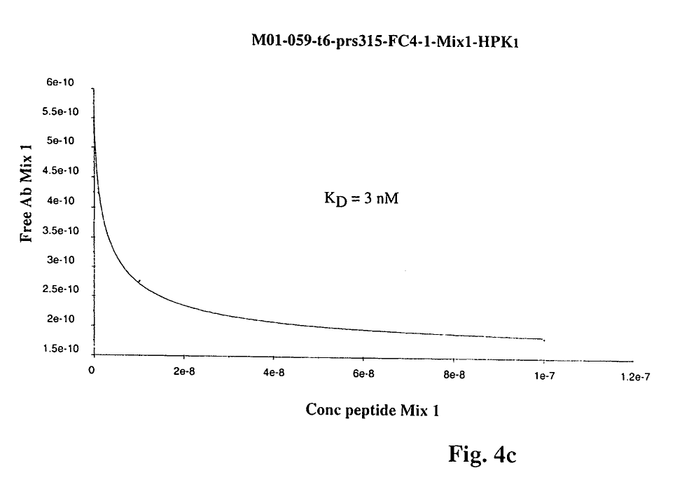

The exact same setting as described above was used. Data from FIGS. 3a to 3c where subgroups of antibodies were injected (0.3 to 20 nM) for 3 min over the surfaces were used to calibrate free antibody concentrations. Then subgroups of free peptides were designed such as only one peptide will bind per analyte (antibody) subgroup. Antibody concentration was fixed to 1 nM in antibody subgroups and various amounts of peptide subgroups were mixed to antibody subgroups. FIGS. 4a to 4c show calculation of the dissociation constants (K_{D}) for 3 molecular pairs based on free antibody expressed against peptide concentration fit with a 4 parameters equation (from the first matrix M01-059 in FIG. 2, with the first antibody-peptide subgroup).

### EXAMPLE 4

Computer-Simulated Assay with Cross-Reactivity

Assume four different analytes, proteins A to D, and four corresponding ligands, antibodies antiA to antiD, where C is 10% cross reactive to antiA. Assume that all antibodies are immobilised at high levels so that the binding is mass transport limited. The binding level of an analyte to a ligand will be influenced by the diffusion properties of the analyte and proportional to the concentration of the analyte provided that the analyte concentration is low. Since the analytes A-D are assumed to be different proteins, they will have different relative diffusion properties as shown below:

Protein A diffuses at a relative rate 1

Protein B diffuses at a relative rate 0.5

Protein C diffuses at a relative rate 0.7

Protein D diffuses at a relative rate 0.7

The flow cell height will also influence the mass transport properties in that flow cell. Suppose that the small differences in the flow cell heights have the following relative influence on the mass transport properties:

Fc1: 1.00

Fc2: 0.95

Fc3: 1.05

Fc4: 1.01

Co-immobilize subgroups of ligand antibodies antiA, antiB, antiC and antiD in the four flow channels (Fc1, Fc2, Fc3, Fc4) as in the sensor chip design shown below.

Fc1: 10 units antiA+8 units antiB

Fc2: 19 units antiB+12 units antiC

Fc3: 7 units antiC+12 units antiD

Fc4: 19 units antiC+14 units antiA

The analyte binding reactions will give the following signals:

protein A binds to antiA giving signal [conc A]*[diffusion rate A]*[relative flow cell specific mass transport properties],

protein B binds to antiB giving signal [conc B]*[diffusion rate B]*[relative flow cell specific mass transport properties],

protein C binds to antiC giving signal [conc C]*[diffusion rate C]*[relative flow cell specific mass transport properties],

protein D binds to antiD giving signal [conc D]*[diffusion rate D]*[relative flow cell specific mass transport properties], and

protein C binds to antiA giving signal [conc C]*0.1*[diffusion rate C]*[relative flow cell specific mass transport properties].

The signal for each flow cell can then be estimated according to:

RU(Fc1)=1.00*(1*[conc A]+0.5*[conc B]+0.7*0.1*[conc C])

RU(Fc2)=0.95*(0.5*[conc B]+0.7*[conc C])

RU(Fc3)=1.05*(0.7*[conc C]+0.7*[conc D])

RU(Fc4)=1.01*(0.7*[conc C]+1*[conc A]+0.7*0.1*[conc C]).

Such a model for the signal from each flow cell can be obtained for any chip by injection of samples with known concentrations of the proteins A, B, C and D. Now, simulate the assay situation as follows:

1. Generate a mix of samples where three of the proteins A, B, C and D have a concentration between 0.1 and 4.1 and the remaining one has a concentration in the range of 0.0001*[0.1 to 4.1]. It is quite important that not all the proteins have a high concentration, otherwise all signals may be messed up (both for matrix and single protein immobilizations).

2. Calculate the RU values for each flow cell and multiply with a random number between 0.95 and 1.05 (to add noise).

3. Use the RU values and the equations for generating the RU values to estimate the concentration.

4. Count the number of successful concentration estimates.

The simple algorithm above was implemented in computer language using MATLAB™ (The MathWorks, Inc., Natick, Mass., U.S.A.) and the simulation was run on a computer. According to the simulation, 84% of the concentration estimates have errors lower than 10%, and 97% of the estimates have errors lower than 30%. This demonstrates that it is possible to have cross-reactive reagents (at least 1 of 4) and still get a fair concentration estimate in a multi-ligand/multi-analyte type approach.

### EXAMPLE 5

Multiple-Ligand/Multiple Analyte Assay

To perform a multivariate assay for the three analytes myoglobin, GST (glutathione S-transferase) and monoclonal IgG1, a sensor chip was sequentially co-immobilized with the antibodies anti-myoglobin, anti-mouse-IgG1 and anti-GST (all the analytes and antibodies were from Biacore AB, Uppsala, Sweden, except monoclonal IgG1, an antibiotin mouse IgG1 obtained from Novocastra Laboratories Ltd., Newcastle upon Tyne, U.K.) according to the following scheme for the different flow cells Fc1 to Fc4 of a BIACORE® 3000-instrument:

Fc1: blank

Fc2: anti-myoglobin (approx. 1400 RU)+anti-IgG1 (approx. 5800 RU)

Fc3: anti-IgG1 (approx. 2000 RU)+anti-GST (approx. 333 RU)

Fc4: anti-GST (approx. 395 RU)+anti-myoglobin (approx. 1350 RU).

Calibration samples were prepared as follows and run through the instrument flow cells:

Myoglobin, 500 ng/ml

Myoglobin, 167 ng/ml

GST, 2000 ng/ml

GST, 666 ng/ml

IgG1 monoclonal, 1000 ng/ml

IgG1 monoclonal, 333 ng/ml.

The results were used to create a mathematical model for the flow cell signals in the corresponding way as described in Example 4 above, but without the cross reactivity terms.

The following “unknown” samples were then prepared and run through the flow cells:

Myoglobin, 250 ng/ml,+GST, 1000 ng/ml,

Myoglobin, 250 ng/ml,+IgG1, 500 ng/ml,

GST, 1000 ng/ml,+IgG1, 500 ng/ml,

GST, 666 ng/ml,+IgG1, 333 ng/ml,+myoglobin, 167 ng/ml.

The concentrations of the analytes in the samples were calculated using the mathematical model.

The results are presented in Tables 5 and 6 below:

CALIBRATIONS (2 replicates of each sample)

| | True value | | |
|---|---|---|---|
| Analyte | ng/ml | Replicate 1 ng/ml | Replicate 2 ng/ml |
| Myo | 500.0 | 463.3 | 501.5 |
| GST | 0.0 | 26.9 | 0.0 |
| IgG1 | 0.0 | 0.0 | 0.0 |
| Myo | 167.0 | 216.6 | 226.7 |
| GST | 0.0 | 0.0 | 0.0 |
| IgG1 | 0.0 | 1.2 | 0.6 |
| Myo | 0.0 | 1.5 | 0.0 |
| GST | 2000.0 | 2001.8 | 1912.5 |
| IgG1 | 0.0 | 0.0 | 0.0 |
| Myo | 0.0 | 5.5 | 0.0 |
| GST | 666.0 | 1203.8 | 1151.3 |
| IgG1 | 0.0 | 17.2 | 8.5 |
| Myo | 0.0 | 0.0 | 2.8 |
| GST | 0.0 | 49.0 | 0.0 |
| IgG1 | 1000.0 | 990.1 | 998.5 |
| Myo | 0.0 | 1.0 | 0.0 |
| GST | 0.0 | 0.0 | 42.7 |
| IgG1 | 333.0 | 388.6 | 376.7 |

TABLE 6
“UNKNOWN” SAMPLES (2 replicates of each sample)

| | True value | | |
|---|---|---|---|
| Analyte | ng/ml | Replicate 1 ng/ml | Replicate 2 ng/ml |
| Myo | 250.0 | 235.6 | 501.5 |
| GST | 1000.0 | 1252.3 | 1253.7 |
| IgG1 | 0.0 | 0.0 | 0.0 |
| Myo | 250.0 | 227.5 | 235.9 |
| GST | 0.0 | 34.5 | 0.0 |
| IgG1 | 500.0 | 353.7 | 354.6 |
| Myo | 167.0 | 241.7 | 247.8 |
| GST | 666.0 | 1340.6 | 1283.7 |
| IgG1 | 333.0 | 343.4 | 334.4 |
| Myo | 0.0 | 8.9 | 3.9 |
| GST | 1000.0 | 1179.6 | 1145.2 |
| IgG1 | 500.0 | 362.1 | 367.9 |

The mathematical model is able to deconvolute analyte signals from multi-ligand surfaces when the system is mass transport limited as demonstrated above for the analytes myoglobin and IgG1. Some values for GST were overestimated because of a non-linear relationship between signal level and concentration, due to a poor immobilization level of anti-GST antibodies.

From the foregoing, it will be appreciated that, although specific embodiments of this invention have been described herein for purposes of illustration, various modifications may be made without departing from the spirit and scope of invention. Accordingly, the invention is not limited except by the appended claims.

1. An assay method, comprising:
(a) providing a plurality of discrete solid support surface areas,
(b) providing a plurality of different ligands,
(c) defining a first set of different groups of the plurality of ligands,
(d) immobilizing each group of ligands on a different solid support surface area,
(e) providing a plurality of different analytes, each of which is capable of binding to a respective one of the plurality of ligands, at least a major part of the analytes having substantially no cross-reactivity to other ligands,
(f) defining a first set of different groups of the plurality of analytes, each analyte being present in at least one group,
(g) sequentially contacting each group of analytes with the surface areas to bind the analytes in each group to immobilized ligands, and
(h) detecting the interaction of each group of analytes with each group of ligands to determine therefrom the amount of binding of each analyte.

2. The method according to claim 1, wherein at least about 75%, preferably at least about 90% of the analytes, bind specifically to a respective one of the plurality of ligands.

3. The method according to claim 1, wherein each analyte binds specifically to a respective one of the plurality of ligands.

4. The method according to claim 1, wherein none of the different groups of ligands includes all the different ligands.

5. The method according to claim 1, wherein none of the different groups of analytes includes all the different analytes.

6. The method according to claim 1, wherein each ligand is present in at least two different groups of ligands.

7. The method according to claim 1, wherein the groups of ligands and the groups of analytes are defined such that in each group of analytes, each analyte binds specifically to a different one of the different groups of ligands.

8. The method according to claim 1, wherein steps e) to h) in claim 1 are repeated with a second set of different groups of analytes, differently defined than the first set, to determine the possible influence of other analytes on the binding of a specific analyte to a specific ligand.

9. The method according to claim 1, wherein steps b) to h) in claim 1 are repeated with a second set of different groups of ligands, differently defined than the first set, to determine the possible influence of other ligands on the binding of a specific analyte to a specific ligand.

10. The method according to claim 1, wherein each group of analytes contains at least three different analytes.

11. The method according to claim 1, wherein each group of ligands contains at least three different ligands.

12. The method according to claim 7, which comprises providing a plurality of soluble ligands or ligand analogues which bind specifically to respective ones of the analytes, defining different groups of the soluble ligands or ligand analogues such that one ligand or ligand analogue in each group thereof binds specifically to one analyte in each group of analytes, and prior to step g) in claim 1 mixing each group of ligands or ligand analogues with its respective group of analytes.

13. The method according to claim 7, wherein prior to step g) in claim 1, each group of analytes is mixed with binding agents that compete with the analytes for the binding to their respective immobilized ligands.

14. The method according to claim 7, wherein prior to step g) in claim 1, respective specific binding partners to the immobilized ligands are contacted with the different solid support surface areas.

15. The method according to claim 1, wherein after determining the binding of the analytes in a group in step h) in claim 1, the surface areas are contacted with a regeneration solution, and the capability of the regeneration solution to remove each analyte from its ligand is determined.

16. The method according to claim 15, wherein the surface areas subjected to regeneration solution are sequentially contacted with the different groups of analytes to determine any change in binding in relation to that determined in step h) in claim 1.

17. The method according to claim 15, which is repeated with at least one different regeneration solution.

18. The method according to claim 1, wherein the solid support areas are sensing surface areas.

19. The method according to claim 1, wherein the interactions at the surface are monitored in real time.

20. The method according to claim 1, wherein mass changes at the surface areas are detected.

21. The method according to claim 1, wherein the detection is based on evanescent wave sensing.

22. The method according to claim 1, wherein the detection is based on surface plasmon resonance (SPR)

23. The method according to claim 18, wherein the sensing surface areas are provided in at least one flow cell.

24. The method according to claim 1, which comprises determining at least one of ligand immobilization efficiency, analyte concentration, interaction affinity and interaction kinetics.

25. An assay method comprising:
(a) providing a plurality of discrete solid support surface areas,
(b) providing a plurality of different ligands,
(c) defining different groups of the plurality of ligands,
(d) immobilizing each group of ligands on a different solid support surface area,
(e) providing a plurality of different analytes, each analyte having a first binding site and a second binding site, wherein the first binding site of each analyte is capable of binding specifically to a respective one of the plurality of ligands, at least a major part of the first binding sites having substantially no cross-reactivity to other ligands,
(f) contacting each surface area with the analytes to bind the analytes to the immobilized ligands,
(g) providing a plurality of different reagents capable of binding to a respective one of the plurality of analytes at the second binding site thereof, at least a major part of the reagents having substantially no cross-reactivity to other analytes,
(h) defining different groups of the plurality of reagents,
(i) sequentially contacting the surface areas with each group of reagents to bind the reagents in each group to ligand-bound analytes, and
(j) detecting the interaction of each group of reagents with each group of ligand-bound analytes to determine therefrom the amount of binding of each analyte.

26. The method according to claim 25, wherein at least about 90% of the total of analytes and reagents have substantially no cross-reactivity.

27. The method according to claim 25, wherein each analyte is capable of binding specifically to a respective one of the plurality of ligands.

28. The method according to claim 25, wherein each reagent is capable of binding specifically to a respective one of the plurality of analytes.

29. The method according to claim 25, wherein each ligand is present in at least two different groups of ligands.

30. The method according to claim 25, wherein the groups of ligands and the groups of reagents are defined such that in each group of reagents, each reagent binds specifically to a different one of the different groups of immobilized ligands having analytes bound thereto.

31. The method according to claim 25, wherein the solid support areas are sensing surface areas, and the detection is based on evanescent wave sensing.

32. The method according to claim 25, which comprises determining at least one of ligand immobilization efficiency, analyte concentration, interaction affinity and interaction kinetics.

33. An assay method comprising:
(a) providing a plurality of discrete solid support surface areas,
(b) providing a plurality of n different ligands, wherein n is at least 2,
(c) defining different groups of the plurality of n ligands comprising single ligands and combinations of from two to n different ligands,
(d) immobilizing each group of ligands on a different surface area,
(e) sequentially contacting a plurality of n different analytes with each surface area, at least a major part of the analytes being capable of specifically binding to a respective one of the plurality of different ligands, and
(f) detecting the interaction of each analyte with each group of ligands to determine therefrom the amount of ligand-binding of each analyte, and the possible influence of ligand-ligand interaction on the binding of analyte to immobilized ligand.

34. The method according to claim 33, wherein at least about 75%, preferably at least about 90% of the analytes, bind specifically to a respective one of the plurality of ligands.

35. The method according to claim 33, wherein each analyte binds specifically to a respective one of the plurality of ligands.

36. The method according to claim 33, wherein in step e) in claim 33, the surface areas are sequentially contacted with different groups of analytes, comprising single analytes and combinations of from two to n different analytes.

37. The method according to claim 33, wherein the solid support areas are sensing surface areas, and the detection is based on evanescent wave sensing.

38. The method according to claim 33, which comprises determining at least one of ligand immobilization efficiency, analyte concentration, interaction affinity and interaction kinetics.

39. An assay method comprising:
(a) providing a plurality of discrete solid support surface areas,
(b) providing a plurality of n different ligands, wherein n is at least 2,
(c) defining different groups of the plurality of n ligands comprising single ligands and combinations of from two to n different ligands,
(d) immobilizing each group of ligands on a different surface area,
(e) contacting each surface area with a plurality of n analytes to bind them to the ligands, at least a major part of the analytes being capable of binding through a first binding site thereof specifically to a respective one of the plurality of different ligands,
(f) sequentially contacting a plurality of n different reagents with each surface area, at least a major part of the reagents being capable of specifically binding to a respective one of the plurality of different analytes via a second binding site thereof, and
(g) detecting the interaction of each reagent with its ligand-bound analyte to determine therefrom the amount of ligand-binding of each analyte, and the possible influence of ligand-ligand interaction on the binding of analyte to immobilized ligand.

40. The method according to claim 39, wherein at least about 90% of the total of analytes and reagents have substantially no cross-reactivity.

41. The method according to claim 39, wherein each analyte is capable of binding specifically to a respective one of the plurality of ligands.

42. The method according to claim 39, wherein each reagent is capable of binding specifically to a respective one of the plurality of analytes.

43. The method according to claim 39, wherein each ligand is present in at least two different groups of ligands.

44. The method according to claim 39, wherein the groups of ligands and the groups of reagents are defined such that in each group of reagents, each reagent binds specifically to a different one of the different groups of immobilized ligands having analytes bound thereto.

45. The method according to claim 39, wherein in step f) in claim 39, the surface areas are sequentially contacted with different groups of the reagents, comprising single reagents and combinations of from two to n different reagents.

46. The method according to claim 39, wherein the solid support areas are sensing surface areas, and the detection is based on evanescent wave sensing.

47. The method according to claim 39, which comprises determining at least one of ligand immobilization efficiency, analyte concentration, interaction affinity and interaction kinetics.

48. An assay method comprising:
(a) providing a plurality of discrete solid support surface areas, (b) providing a plurality of different ligands, (c) defining different groups of the plurality of ligands, (d) immobilizing each group of ligands on a different solid support surface area,
(e) providing a plurality of different analytes, each of which is capable of binding to a respective one of the plurality of ligands, at least a major part of the analytes having substantially no cross-reactivity to other ligands,
(f) sequentially contacting each analyte with the surface areas to bind the analytes to the immobilized ligands, and
(g) detecting the interaction of analyte with each group of immobilized ligands to determine therefrom the amount of binding of each analyte.

49. The method according to claim 48, wherein at least about 75%, preferably at least about 90% of the analytes, bind specifically to a respective one of the plurality of ligands.

50. The method according to claim 48, wherein each analyte binds specifically to a respective one of the plurality of ligands.

51. The method according to claim 48, wherein the solid support areas are sensing surface areas, and the detection is based on evanescent wave sensing.

52. The method according to claim 48, which comprises determining at least one of ligand immobilization efficiency, analyte concentration, interaction affinity and interaction kinetics.

53. An assay method comprising:
(a) providing a plurality of discrete solid support surface areas,
(b) providing a plurality of different ligands,
(c) defining different groups of the plurality of ligands,
(d) immobilizing each group of ligands on a different solid support surface area,
(e) providing a plurality of different analytes, each analyte having a first binding site and a second binding site, at least a major part of the analytes being capable of binding through the first binding site specifically to a respective one of the different ligands,
(f) contacting each surface area with the analytes to bind the analytes to the immobilized ligands,
(g) providing a plurality of different reagents, at least a major part of the reagents being capable of binding specifically to a respective one of the plurality of analytes at the second binding site thereof,
(h) sequentially contacting each reagent with the surface areas to bind the reagents to the ligand-bound analytes, and
(j) detecting the interaction of each reagent with each surface area to determine therefrom the amount of binding of each analyte.

54. The method according to claim 53, wherein at least about 90% of the total of analytes and reagents have substantially no cross-reactivity.

55. The method according to claim 53, wherein each analyte is capable of binding specifically to a respective one of the plurality of ligands.

56. The method according to claim 53, wherein each reagent is capable of binding specifically to a respective one of the plurality of analytes.

57. The method according to claim 53, wherein each ligand is included in at least two different groups of ligands.

58. The method according to claim 53, wherein the ligands are antibodies and each subgroup of ligands contains different clones of the same antibodies.

59. The method according to claim 53, wherein the solid support areas are sensing surface areas, and the detection is based on evanescent wave sensing.

60. The method according to claim 53, which comprises determining at least one of ligand immobilization efficiency, analyte concentration, interaction affinity and interaction kinetics.

61. A method of determining regeneration conditions for ligand-supporting solid support surfaces, comprising:
(a) providing a plurality of discrete solid support surface areas,
(b) providing a set of a plurality of different ligands,
(c) immobilizing the set of ligands on at least two different surface areas,
(d) sequentially contacting each surface area with a plurality of analytes, at least a major part of the analytes being capable of specifically binding to a respective one of the different ligands,
(e) determining the interaction of each analyte with each surface area having immobilized ligands thereon,
(f) subjecting each surface area having the analytes bound to immobilized ligands to a different regeneration solution,
(g) sequentially contacting the plurality of analytes with each surface area, and
(h) determining for each analyte any change in analyte binding in relation to the binding determined in step e).

62. The method according to claim 61, wherein at least about 75%, preferably at least about 90% of the analytes, bind specifically to a respective one of the plurality of ligands.

63. The method according to claim 61, wherein each analyte binds specifically to a respective one of the plurality of ligands.

64. The method according to claim 61, wherein the solid support areas are sensing surface areas, and the detection is based on evanescent wave sensing.
